(19) United States
(12) Patent Application Publication
(10) Pub. No.: US 2022/0117181 A1
(43) Pub. Date: Apr. 21, 2022

(54) Method for Breeding Cannabis Cultivars Based on Epigenetic Regulation

(71) Applicant: Mark Jasinski, Traverse City, MI (US)

(72) Inventor: Mark Jasinski, Traverse City, MI (US)

(21) Appl. No.: 17/075,858

(22) Filed: Oct. 21, 2020

(51) Int. Cl. A01H 1/06; A01G 15/00; A01G 24/10

(52) U.S. Cl. CPC A01H 1/06; A01G 24/10; A01G 15/00

(57) ABSTRACT

A method alters genetic material of a plant, which is capable of growing flower buds, when grown from soil and in an atmosphere. The method includes modifying the soil to have a pH below 7.0. The method also includes the step of eliminating free oxygen in the atmosphere when the flower buds of the plant become visible.

### BACKGROUND ART

### 1. Field of the Invention

The method relates to the regulation of sequences of DNA, RNA and proteins. Particularly, the method relates to downregulating THCA and upregulating CBGA, CBDA and terpenes in Cannabis species.

### 2. Description of the Related Art

Cannabinoids are complex molecules unique to Cannabis species. The cannabinoids Delta-9-Tetrahydrocanabadiolic acid (THCA), Cannabidiolic acid (CBDA) and Cannabigerolic acid (CBGA) are activated through non-enzymatic decarboxylation to form Tetrahydrocannabinol (THC), Cannabidiol (CBD) and Cannabigerol (CBG).

Geranyl pyrophosphate and Olivetolic acid are enzymatically converted to CBGA in the first step of the cannabinoid biosynthetic pathway. In general, cannabinoids are classified as prenylated polyketides.

THCA and CBDA are derived from CBGA, thus accumulation of either molecule (THCA, CBDA) is affected by competition for a shared precursor molecule. Competition for a single precursor (CBGA) leads to inefficiency in production of CBDA; with the elimination of THCA, CBDA production is upregulated by an increase in pathway flux.

Decarboxylation of THCA, CBDA and CBGA is achieved through exposure to light or heat. This process, as well as detection and quantification of each compound by HPLC-based (High-performance liquid chromatography) methods, is simplified by elimination or dramatic downregulation of THCA.

Molecular oxygen is required to synthesize THCA and CBDA from CBGA enzymatically. This process involves flavinylated (“activated”) FAD (flavin adenine dinucleotide) as an electron donor and acceptor in consecutive steps. Simply put, flavinylated FAD acts as an electron shuttle, transporting electrons from one molecule to another. Hydrogen peroxide is produced as a result of FAD transferring two electrons to molecular oxygen.

THCA, CBDA and CBGA are secreted from the trichome in Cannabis species. Trichomes are glandular, hair-like organs which are implicated in defense response in Cannabis, as THCA, CBDA and CBGA are toxic to the plant through their shared ability to participate in oxidation reactions.

### SUMMARY OF THE INVENTION

A method alters genetic material of a plant, which is capable of growing flower buds, when grown from soil and in an atmosphere. The method includes modifying the soil to have a pH below 7.0. The method also includes the step of eliminating free oxygen in the atmosphere when the flower buds of the plant become visible.

### DETAILED DESCRIPTION

Definitions

Base Pair: a pair of complementary bases in a double-stranded nucleic acid molecule, consisting of a purine in one strand linked by hydrogen bonds to a pyrimidine in the other. Cytosine always pairs with guanine, and adenine with thymine (in DNA) or uracil (in RNA)

Single Nucleotide Polymorphism/Single Nucleotide Variant (SNP/SNV): A base pair of DNA, RNA or protein at a specific position in the sequence of interest (Numbered/ordered accordingly with reference sequence) that differs from the base pair shown in the reference sequence. In the example below, the base pair “A” at position 3 in the Sequence of Interest is deemed as a SNP or SNV, as it differs from the base pair “G” in the Reference Sequence.

Position: 1 2 3 4 5

Sequence of Interest: A A A AA

Reference Sequence: A A G AA

Marker: a position in the genome that is used to track genes throughout multiple generations.

Population: any number of progenies derived from a cross between two parents.

Sequence: DNA of a given organism, individual base pairs may be ordered and numbered in a method which is useful for comparison.

Significant: Unless denoted otherwise, the term “significant” refers to a difference between any values under comparison, validated by a statistic test which renders a p-value less than 0.05.

Epigenetic: Of a stable, heritable, measurable phenotypic trait; a change or changes to said trait, which are not a result of an alteration in DNA sequence.

Genetic: Of a stable, heritable, measurable phenotypic trait; a change or changes to said trait, which are a result of an alteration in DNA sequence.

Laterite: Of soil; rich in ferrous oxide (Iron oxide, chemical formula: FeO; typically red-yellow in color), composed of up to 50% by weight of iron oxides; iron and aluminium oxides; iron, aluminium and titanium oxides; or iron, aluminium, titanium and manganese oxides.

10-10 −10 Fertilizer: Fertilizer which includes, by weight, 10% nitrogen (chemical formula: N), 10% phosphorus-containing compound, typically phosphate (chemical formulas P and P₂O₅, respectively) and 10% potassium-containing compound, typically potassium oxide (chemical formulas K and K₂O, respectively), with the remainder being soil.

15-15 −15 Fertilizer: Fertilizer which includes, by weight, 15% nitrogen (chemical formula: N), 15% phosphorus-containing compound, typically phosphate (chemical formulas P and P₂O₅, respectively) and 15% potassium-containing compound, typically potassium oxide (chemical formulas K and K₂O, respectively).

### MATERIALS AND METHOD

Plant Material

Plant material is selected to maximize variability in THCA, CBDA, CBGA, terpenes, plant trait(s) of interest among accessions, as will be discussed in greater detail below.

Cultivation Method

Grow and maintain plants in laterite soil, climate-controlled location (oxygen and carbon dioxide levels in particular), under multiple light sources having wavelengths in the range between 380-750 nm, adjusting day/night light proportions based on flowering requirements for individual cultivars if necessary. Cultivating the same plants in an outdoor location will assist in genetic mapping, through comparison, if grower chooses to utilize this method during cultivation.

During vegetative stage of plant growth (principle growth stages 1-4), the humidity is held between 75-95%; the oxygen and CO₂ levels are held in a 1:1 ratio; and the temperature is held below 61° F. for one to three days. Gradually raise temperature to 92° F. to promote seed imbibition, as well as vegetative growth.

After the vegetative stage, the plant enters the flowering stage, defined as principle growth stage 5, when the first visible individual flower buds appear. At this stage, the humidity is lowered to 60-75% and the temperature is lowered to below 81° F.

Upon appearance of a plurality of flower buds (Principle Growth stage 55-5), the humidity is lowered again to a level between 50-60%. The temperature is also reduced a subsequent time to approximately 71° F. All free oxygen is removed from the environment for remainder of the method. Therefore, the remainder of the method is performed in an anoxic atmosphere.

THCA content is sampled continuously. If THCA production is nearing an unacceptable level, the plant is harvested. If there are any signs of plant deterioration, the plant is harvested. In many instances the entire crop to which the plant is a part is also harvested.

The plants are phenotyped for THCA, CBDA, CBGA, terpene content, trait(s) of interest. If the plants express the desired traits, the method proceeds to the step of locating base pairs showing evidence of epigenetic modification, discuss in greater detail subsequently.

A cultivation method may be used that begins with equal periods of light and dark settings. During the vegetative state of growth, the daily ratio of light to dark is switched to 3:1. To induce flowering (Principle growth stage 5—First individual flower buds of flowers visible), the daily ratio of light to dark is reverted back to 1:1. This ratio may be adjusted based on performance of strain(s). Upon the appearance of the bulk of flower buds (Principle Growth stage 55-5), the daily light/dark ratio is again set to 3:1, with the possibility of the daily ratio being adjusted even higher based on the performance of strain(s).

Location of Base Pairs Showing Evidence of Epigenetic Modification—to be Completed oncurrent to Cultivation Method

While performing the cultivation method set forth above, specimens of the cultivars can be tested to evidence epigenetic modifications therein. Whole genome sequences, or sequences of genetic region of interest for all test accessions/subjects can be obtained. This can be achieved using any type of sequencing known to those skilled in the art.

All accessions/subjects are phenotyped for content of molecule/compound of interest (e.g., CBDA) over span of at least two generations or two time points. Weather and environmental data may assist in the identification of environmental factors inducing epigenetic modifications. Obtaining phenotypic data at different developmental stages of organism's lifespan may assist in downstream mapping of phenotypic data to genetic base pair(s) significantly associated with production of molecule/compound of interest. (Example: Plant #77 produced 50.34 g of CBD in 2019 and 34.76 g of CBD in 2020).

Sequenced reads are processed using software tools known to those skilled in the art. The sequenced reads are aligned to a reference genome, if applicable. If no reference genome exists, a library is created using preferred methods of de novo sequence alignment. (Example: Sequenced reads were processed using the default parameters of TASSEL 4.0 GBS Pipeline. Sequenced reads were aligned to Cannabis reference genome, acquired from NCBI.)

Quality-control/SNP filtering methods are performed (removal of SNPs with low genotyping rate, removal of SNPs with minor allele frequency below x % etc.) to remove potential accessions/subjects, SNPs with low genotyping rate and to filter SNPs based on the goal of the individual study. Enough SNPs are retained to assure adequate coverage (at least 1 SNP per 2500 base pairs in sample library genome) and SNPs with minor allele frequency between 1-5% are suggested (filter SNPs with lower than 0.0025-0.01% minor allele frequency based on population size). Linkage disequilibrium filtering is not performed. (Example: SNP filtering was performed in PLINK v1.9 (www.coggenomics.org/plink/1.9/; Chang et al., 2015). SNPs with a genotyping rate below 90% were removed from the study, along with accessions with lower than a 90% genotyping rate. The subset of SNPs used in analysis was obtained by removing SNPs with minor allele frequency (MAF) lower than 1%. Having the MAF set to be greater than 1% was considered stringent enough to remove sequencing errors but retain rare alleles. Linkage disequilibrium filtering was not applied).

Perform Association Analysis using preferred Genome-Wide Association (GWA) program (such as TASSEL, MAGMA or FarmCPU) and population correction/stringency methods (GLM, MLM, principal component analysis etc.), using subset of SNPs acquired in step above. It is suggested that each phenotypic trait be analyzed separately, rather than analysis of every trait in one run. A significant cut-off point for individual SNPs may be determined based on goals of individual study. (Example, association analyses were performed using TASSEL v5.0. A principal component matrix was constructed from principal component analysis (PCA) using both phenotypic and genotypic data from all accessions and was used to correct for population structure. In TASSEL, a General Linear Model using PCA to correct for population structure (GLM-PCA) was built individually for each phenotypic trait. SNPs were deemed significant in TASSEL if their calculated p-value was below the threshold applied using the Bonferroni Multiple Tests correction at P<0.05, thus suggesting association between the marker and the phenotypic trait being tested).

If appropriate, examine individual SNPs in data visualization software, such as the NCBI Genome Browser, which can visually provide data, such as the position of SNP on a particular chromosome, the nature of a SNP in relation to a gene, whether a particular sequence has been alternatively spliced, and published RNA-seq data (Data showing expression of given stretch of DNA).

If a given SNP lies in a stretch of uncharacterized DNA, a preferred local prediction tool is used to attempt in finding a predicted sequence to characterize the given SNP in relation to. Basic local alignment search tool (BLAST-NCBI local prediction tool) is the recommended local prediction tool. RNA-seq data can be useful at this point in showing if previous gene expression in the region of the SNP has ever been published.

Any base pair(s) which differs between the two sequences of individual accessions/subjects at a given position (i.e. Plant #1: Sequence obtained in generation #1, sequence obtained in generation #2) may be considered as a candidate for epigenetic regulation. All SNPs can be considered as candidates for genetic regulation

Usage of Markers in Breeding

Select significant markers shown to be associated with upregulated and downregulated traits of interest in plants.

Design breeding crosses with markers selected in the above process to downregulate THCA content in plants of interest. This step is repeated until THCA measured in plants of interest is downregulated to 0.299% of plant mass or lower.

Likewise, design breeding crosses with markers selected in the above process to upregulate CBDA, CBGA, compound(s) of interest to extent preferred, repeating crosses as desired

The process has been described in an illustrative manner. It is to be understood that the terminology, which has been used, is intended to be in the nature of words of description rather than of limitation.

Many modifications and variations of the method are possible in light of the above teachings. Therefore, within the scope of the appended claims, the invention may be practiced other than as specifically described.

I claim:

1. A method for altering genetic material of a plant, capable of growing flower buds, grown from soil in an atmosphere, the method comprising the steps of:
modifying the soil to have a pH below 7.0; and
eliminating free oxygen in the atmosphere when the flower buds of the plant become visible.

2. A method as set forth in claim 1 wherein the step of modifying the soil includes augmenting the soil such that it contains up to 50% by weight of material from a group of: iron oxides; iron and aluminium oxides; iron, aluminium and titanium oxides; and iron, aluminium, titanium, and manganese oxides.

3. A method as set forth in claim 1 including the step of humidifying the atmosphere to a range between 75% and 95% humidity.

4. A method as set forth in claim 3 including the step of raising the temperature of the atmosphere to greater than 90° F. during a vegetative state of the plant.

5. A method as set forth in claim 4 including the step of first reducing the temperature when a first of the flower buds of the plant become visible.

6. A method as set forth in claim 5 wherein the step of first reducing the temperature includes reducing the temperature to approximately 81° F.

7. A method as set forth in claim 4 including the step of first reducing the humidity of the atmosphere when the first of the flower buds of the plant become visible.

8. A method as set forth in claim 7 wherein the step of first reducing the humidity includes first reducing the humidity to 60% to 75%.

9. A method as set forth in claim 7 including the step of subsequently reducing the temperature when a plurality of flower buds of the plant become visible.

10. A method as set forth in claim 9 wherein the step of subsequently reducing the temperature includes reducing the temperature to approximately 71° F.

11. A method as set forth in claim 9 including the step of subsequently reducing the humidity of the atmosphere when the plurality of flower buds of the plant become visible.

12. A method as set forth in claim 11 wherein the step of subsequently reducing the humidity includes subsequently reducing the humidity to 50% to 60%.
